(19) United States
(12) Patent Application Publication
(10) Pub. No.: US 2014/0242603 A1
(43) Pub. Date: Aug. 28, 2014

(54) METHODS AND APPARATUS FOR TARGET CELL MAGNETIC ENRICHMENT, ISOLATION AND BIOLOGICAL ANALYSIS

(71) Applicants: Glenn Yaguang Deng, San Jose, CA (US); Jing Zhang, Los Altos, CA (US); Brian Deng, San Jose, CA (US)

(72) Inventors: Glenn Yaguang Deng, San Jose, CA (US); Jing Zhang, Los Altos, CA (US); Brian Deng, San Jose, CA (US)

(21) Appl. No.: 14/181,671

(22) Filed: Feb. 15, 2014

Related U.S. Application Data
(60) Provisional application No. 61/769,195, filed on Feb. 26, 2013.

(51) Int. Cl. G01N 1/40; C12Q 1/24

(52) U.S. Cl. CPC G01N 1/4077; C12Q 1/24
USPC 435/7.1; 435/30; 435/40.5

(57) ABSTRACT

Disclosed here are methods and apparatus for target cell magnetic enrichment, isolation, and biological analysis. The target cells are labeled with magnetic nano-particles in a separate tube, container or in a device with soft magnetic collectors but not activated during the labeling period. Labeled biological samples will be used for targets enrichment and isolation using a magnetic device (permanent magnet or electromagnet). The labeled sample passes through a tube/channel within a magnetic field so the magnetic nano-particle labeled targets can be captured. Once the magnetic field is turned off, the captured targets can be released in a container. The labeling of the targets also can be performed in a device with soft magnetic collectors inside. Without magnetic activation, the device is just like a regular biological sample incubator. Thus the targets are labeled during the mixing and incubation. Once the device is placed in the magnetic field, the soft magnetic collectors are activated and the labeled target cells are isolated. After the washing process, the pure targets are released into the target collector by turning off the magnetic field with or without centrifugation. The magnet can be a permanent magnet or an electromagnet producing the magnetic field for targets to bind to the soft magnetic collectors in the device. The soft magnetic collectors can function as a magnet for capturing and releasing targets without the need of separation covers between magnet and target biological structures. The methods and apparatus improve the targets capturing sensitivity which improves the operation of the device. The methods and apparatus can be used for enrichment, isolation and material transferring.

### CROSS REFERENCE OF RELATED APPLICATIONS

The present application claims the benefit of U.S. Provisional Application No. 61/769,195, filed on Feb. 26, 2013, which is hereby incorporated by reference in its entirely.

### STATEMENT REGARDING FEDERALLY SPONSORED RESEARCH OR DEVELOPMENT

None

### BACKGROUND OF THE INVENTIONS

1. Field of the inventions

The present invention relates to the methods and apparatus for the enrichment, isolation and biological analysis of target biological materials using permanent or electronic magnets. More specifically, the methods and apparatus transfer the magnetic field from a magnet to soft magnetic collector(s) to let the target biological entities directly bind to the soft magnetic collector(s). This achieves high sensitivity of isolating target biological structure. It simplifies the capturing and releasing process of the biological structures and makes the sensitivity higher and allows for a larger number of samples' process.

2. Background

Common biological markers include specific featured cells such as DNA/RNA, protein and other biological structures in clinics, drug development and basic research. The detection and analyses of these biomarkers with high sensitivity and reproducibility have significant benefits especially in clinics. For example, circulating tumor cells (CTCs) in blood and disseminated tumor cells (DTCs) in bone marrow are considered very valuable biomarkers for cancer patients in clinics. However, these cells are usually present at a low frequency in blood and bone marrow. In most cases it needs technology with high sensitivity and reproducibility to efficiently detect and analyze those cells.

Current methods using indirect magnetic enrichment and isolation of these rare cells lack the sensitivity for routine clinical diagnostics. The sample volumes are also limited by these technologies. Some cell CHIP technology can detect circulating tumor cells with high sensitivity, but it is hard to release the captured cells from it and some magnetic enrichment technology has high purity of captured target cells, but it has to use the support supplies like the plastic sleeve, the sensitivity is reduced then due to the magnet can not directly reach target materials (it has to pass the plastic sleeves). Our invention presents a new method and apparatus that through transferring the magnetic field from a magnet to a differently shaped soft magnetic collector(s) can directly capture and release target biological structures without using plastic sleeves and other support supplies. The method and apparatus have significant higher sensitivity and reproducibility with a greater sample volume capability.

### BRIEF SUMMARY OF THE INVENTION

The following brief summary is not intended to include all features and aspects of the present invention, nor does it imply that the invention must include all features and aspects discussed in this summary.

In certain aspects, the present invention comprises method and apparatus for capturing, isolating and analyzing biological materials including target biological structure labeling, incubation, capturing, washing/cleaning, releasing and collection:

The present invention works by transferring the magnetic field from a permanent magnet or electromagnet to the soft magnetic collector(s) in order to directly capture the magnetic labeled targets. This invention advantageously fills the aforementioned deficiencies by disclosing a method and apparatus in isolating magnetic targets. This invention uses the soft magnetic collector structure(s) inside a container, and uses the permanent magnet or electromagnet outside of the container to transfer the magnetic field pass the well of the container and reach the soft magnetic collector structure inside to directly capture the targets. Targets can be biological cells, DNA/RNA, protein or other biological materials which can be labeled with magnetic nano-particles. Once the magnetic field is activated (magnetized), the soft magnetic collector structure inside of the container will be actively capturing magnetic targets. Once the outside magnetic field is turned off or removed, the targets bound to the soft magnetic collector(s) inside of the container can be released easily. This new invention solves some key problems from current technologies that the device captures targets but finds it hard to release them. Traditional technology can use a strong magnetic field to capture the targets inside of the container without inside soft magnetic collector(s), but all the captured targets get pressed together tightly so the targets cannot be pure or be broken easily, it is not gently and the sensitivity is very low. This invention with desired soft magnetic collector structures inside of the container, the magnetic field can distributed equally so the sensitivity and the purity will be improved.

The present invention disclosed the method and apparatus of target enrichment and isolation with high sensitivity, capable of isolating and collecting targets labeled with small magnetic nano-particles. This invention demonstrated that the same magnet can capture targets through the soft magnetic collector structures, but cannot directly capture the target without the soft magnetic collectors. This demonstrated that by transferring the magnetic field to a soft magnetic collector and letting the targets directly bind to the soft magnetic collectors, the sensitivity will be improved. In another words, the magnetic nano-particles for target labeling can be even smaller than that of current market using. The smaller magnetic nano-particle labeled target cells have advantages in cell identification and downstream analyses.

This invention mechanism will lead to designing new isolation devices. For example, the MagCell-CHIP, Mag-Fluidics allow the labeled or unlabeled biological samples to pass through the MagCell-CHIP or Mag-Fluidics so that target biological samples will be captured gently and efficiently. If the targets are labeled with magnetic nano-particles/microbeads, it will be captured by the activated soft magnetic collector structures inside of a container; if the targets are not labeled, then the target cells can be captured by the magnetic beads/nano-particles conjugated with specific antibodies. The captured targets labeled or unlabeled can be released by turning off the outside magnetic field and collected by precipitation or centrifugation.

The invention provides a method for identifying target materials. One example is target cell staining for cell identification. Once the biological samples enter the MagCell-CHIP or Mag-Fluidics, the targets will be captured and the other non target materials will be removed. The staining reagents can be added to the device to stain the targets for identification purposes. By adding or removing the outside magnetic field, the biological materials can be well mixed and stained equally for better target isolation and identification efficiency.

The purity of the captured targets can be improved. Captured targets inside of the container can be purified by capturing and releasing controlled by magnetic field. By releasing the targets and re-capturing them again after first processing the samples, the trapped target ratio will be reduced significantly.

The process procedure can be easier with this invention method and it will be easier for automation and large number samples process. The sample volume can have a big range by adjusting the device size. The number of samples processed in onetime can be increased significantly because this device does not take much space. The major operation procedures can be done in the container so it can be easier for automation.

Targets washing: Captured target biological materials can be washed several times with appropriate buffer to increase the target purity. It also can be washed without repeating capturing and releasing. The manipulation can be done with various times to ensure easier operation and result quality.

Target cell releasing: The captured targets can be released by turning off the magnetic field. The capturing, washing, and releasing process can be repeated as a procedure or individual steps. All the operation steps can be done manually or automatically.

Released target cells can be transferred to any collection container (tube, collector, channel) for further biological analysis. It is a new technology to release captured targets without any additional efforts and to dissociate the connection between the targets and capture supplies without enzyme digestion of separate sleeves needed.

Target cell biological analyses including morphological and molecular analyses are more reliable. This invention provides the method to use smaller magnetic particles to capture targets which allows the release operation to be gentler and the obtained results are more reliable.

One of the magnetic devices includes one of the magnetic field generators and the tube/channel or soft magnetic collector. Once the magnetic field is turned ON, the labeled target will be captured by the magnetic field once it passes the magnetic field. Once the magnetic field is turned OFF, the captured targets will be released and ready for collection.

One device is the MagCell-CHIP based on this invention. It can be big enough to hold all the samples and finish the labeling process inside or it can be small to collect the targets by enriching the targets from passing through samples. It captures the targets by turning the magnetic field ON. It can also be small so that the labeled sample can pass the MagCell-CHIP and the targets can be captured by the soft magnetic collector under the magnetic field.

Another device is the Mag-Fluidics. The fluidics can be big enough so the sample labeling process can be done inside of the fluidics without a magnetic field. It can also be small so the labeled sample can pass the Mag-Fluidics and the targets can be captured by the soft magnetic collectors inside of the Mag-Fluidics.

The idea of adding soft magnetic collector structures in the device is transferring magnetic fields from a large magnet to small soft magnetic collectors or distributing the magnetic field to a structure so the targets can bind to the soft magnetic collectors. In addition to be able to release it by turning the magnetic field OFF. By directly binding to the soft magnetic collector structures, the targets capturing sensitivity will be significantly increased.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof and where in:

FIG. 1 Inserting a soft magnetic collector to improve the magnet binding capability. In the upper drawing, the magnet and the iron ball can not bind together because the distance; in the lower drawing, by inserting a magnet sensitive metal (iron, co, ni etc.), the magnet binds the inserted soft magnetic collector and also the iron ball (target) binds to the inserted soft magnetic collector. FIG. 1 shows the magnetic field can be transferred from magnet to the inserted soft magnetic collector and reaches to target iron ball. The iron ball here simulates the targets for demonstration and showed the sensitivity was improved by adding the soft magnetic collector.

FIG. 2 represents the schematic diagram of electromagnet to capture and release magnetic nano-particle labeled target cells. The magnetic particles labeled target cells pass the tube/channel in the magnetic field generated by an electromagnet. The target cells will be captured by the upper magnetic pole and none target cells will pass through the magnetic field area. Once the electromagnet turned off, the captured cells will be released from upper of the tube/channel.

FIG. 3 represents the schematic diagram of electromagnet to isolate magnetic microbeads labeled target cells. By moving the tube/channel to the upper magnetic pole or lower magnetic pole, the trapped none target cells will be removed and the purity of the captured target cells will be improved.

FIG. 4 represents the schematic diagram of multiple sample's target cell isolations. Multiple plastic tubes can be set in the magnetic field so it can process multiple biological samples at the same time.

FIG. 5 shows an experiment that magnetic field can be transferred from magnet to a soft magnetic collector to reach the target. The upper shows the iron ball could not be captured in the distance between the permanent magnet and iron ball; The middle shows that by adding a soft magnetic collector results the soft magnetic collector binds to magnet and the target iron ball binds to the soft magnetic collector; The lower shows that it has to increase the distance between the magnet and magnetic target to keep them separated (not bind together). This explains the reason of this technology improves the target capturing sensitivity.

FIG. 6 describes the mechanism of magnetic field activation of soft magnetic collectors that can let the magnetic target bind to the soft magnetic collector (magnetic insert).

FIG. 7 describes the mechanism of a soft magnetic collector can be bound between two magnets with different magnetic field direction.

FIG. 8 describes the mechanism of this invention that the magnetic target can bind by two magnets in different magnetic field directions.

FIG. 9 shows an experiment that an iron ball can be bound to two permanent magnets in different magnetic field directions.

FIG. 10 illustrates a method to enrich and isolate target cells but remove the non target cells.

FIG. 11 illustrates a method to enrich and isolate target cells but remove the non target cells.

FIG. 12 illustrates a method to enrich and isolate target cells but remove the non target cells.

FIG. 13 illustrates a method to enrich and isolate target cells but remove the non target cells.

FIG. 14 shows an electromagnet device to isolate magnetic microbeads labeled target cells. With the repeats of capturing and releasing and re-capturing the targets from sample, it can improve the target purity.

FIG. 15 shows the captured target cells in zoomed observation.

FIG. 16 shows experiment that a magnetic device for target cell isolation with enlarged observation.

FIG. 17 shows magnetic microbeads labeled target cells captured by electronic magnet.

FIG. 18 shows schematic design to transfer the magnetic field from large magnet to a small soft magnetic collector to capture the target.

FIG. 19 shows an experiment that magnetic field can pass through the plastic well and transfer to the soft magnetic collector to capture the target. Without magnetic insert between the magnet and target, in a certain distance and the plastic well, the target may not be captured, but by adding an insert soft magnetic collector between the magnet and target, the target can be captured and the sensitivity will be improved than that without the inserted soft magnetic collector.

FIG. 20 illustrates a design of MagCell-CHIP that the magnetic post inside of the plastic container (box), the treated biological samples can enter the CHIP, so the target cells will be captured by the magnetic post under certain level of the magnetic field charge, the non target cells will be removed.

FIG. 21 shows the works of MagCell-CHIP capturing or releasing the targets.

FIG. 22 illustrates the procedure that the target cells will be captured and the non target cells will be removed using the MagCell-CHIP.

FIG. 23 illustrates the procedure that the target cells will be captured and the non target cells will be removed using the MagCell-CHIP.

FIG. 24 illustrates the pure target cells are captured after washing process using the MagCell-CHIP.

FIG. 25 illustrates the target cells are released and are collected using the MagCell-CHIP.

FIG. 26 illustrates a MagCell-CHIP that the sample can enter the CHIP from different direction.

FIG. 27 illustrates the captured target cells can be stained within the MagCell-CHIP.

FIG. 28 illustrates the captured target cells can be treated with enzyme to remove any linkers between the magnetic beads and the target cells inside of the MagCell-CHIP.

### DETAILED DESCRIPTION OF THE DRAWINGS

Referring the drawings wherein the showings are for purposes of illustrating a preferred embodiment of the invention only and not for purposes of limiting the applications.

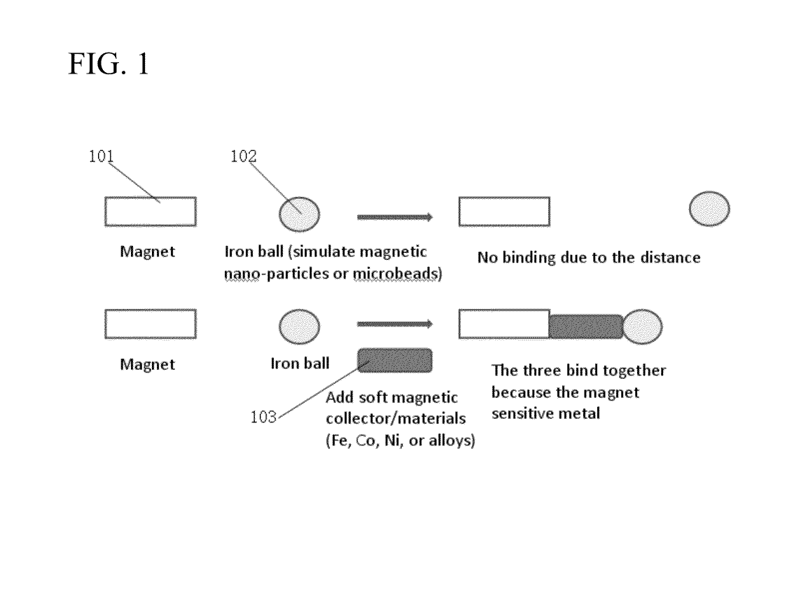

FIG. 1 shows one of the principle of the invention that adding a soft magnetic collector can improve the magnet binding capability in distance. In the upper drawing, the magnet 101 and the iron ball 102 can not bind together due to the distance; in the lower drawing, by inserting a soft magnetic collector 103 (Fe, Co, Ni, or alloys), the magnet binds the inserted soft magnetic collector and also the iron ball (is to simulate magnetic nano-particles or microbeads or labeled targets) binds to the inserted soft magnetic collector. FIG. 1 shows the magnetic field can be transferred from magnet to the inserted soft magnetic collector and reach to target iron ball.

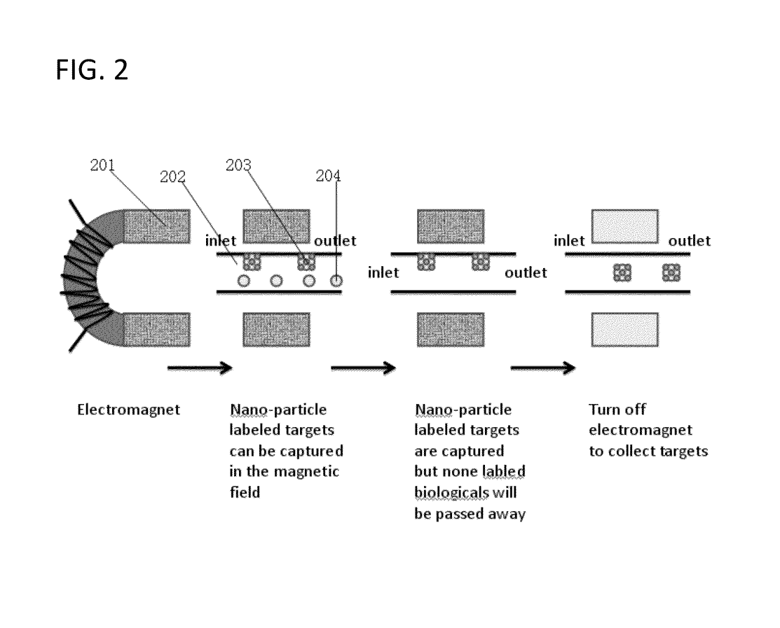

FIG. 2 represents the schematic diagram of electromagnet 201 to capture and release magnetic microbeads labeled target cells 203. The magnetic particles labeled target cells pass the plastic tube 202 in the magnetic field generated by an electromagnet. The target cells will be captured to the upper and non target cells 204 will be passed through and be removed. Once the electromagnet turned OFF, the captured cells will be released from upper of the tube. This describes the electromagnet for target cell enrichment and isolation.

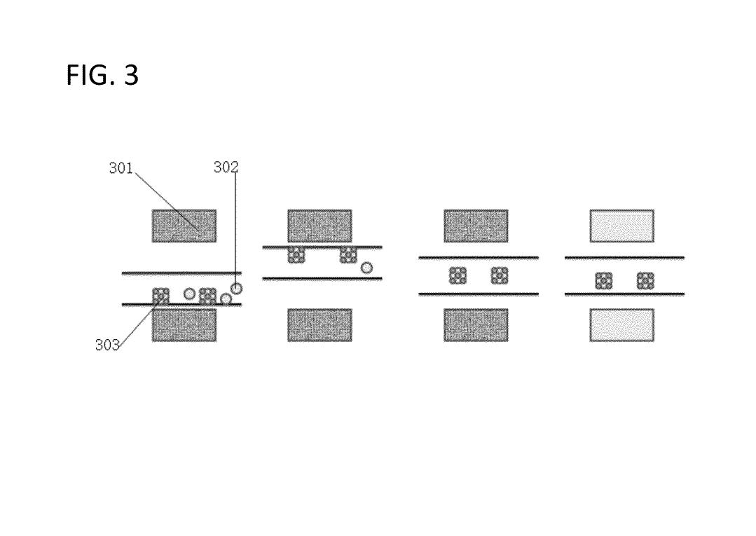

FIG. 3 represents the schematic diagram of electromagnet 301 to isolate magnetic nano-particle labeled target cells 303. By moving the tube/channel to the upper magnetic pole or lower magnetic pole, the trapped non target cells 302 will be removed and the purity of the captured target cells will be improved. This describes that the device for reducing the trapped non targets and improve the target purity by modifying the operation procedures.

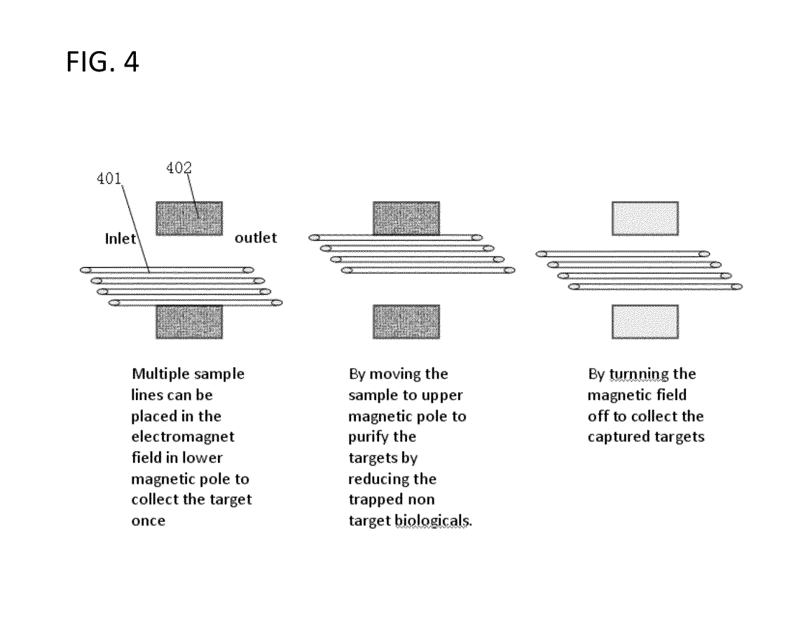

FIG. 4 represents the schematic diagram of multiple sample's target cell isolations. Multiple tubes/channels 401 can be set up in the magnetic field so it can process multiple biological samples at the same time under electromagnet 402 control, alternatively, the same goal can be achieved by controlling electrical current thru electromagnet w/o moving samples.

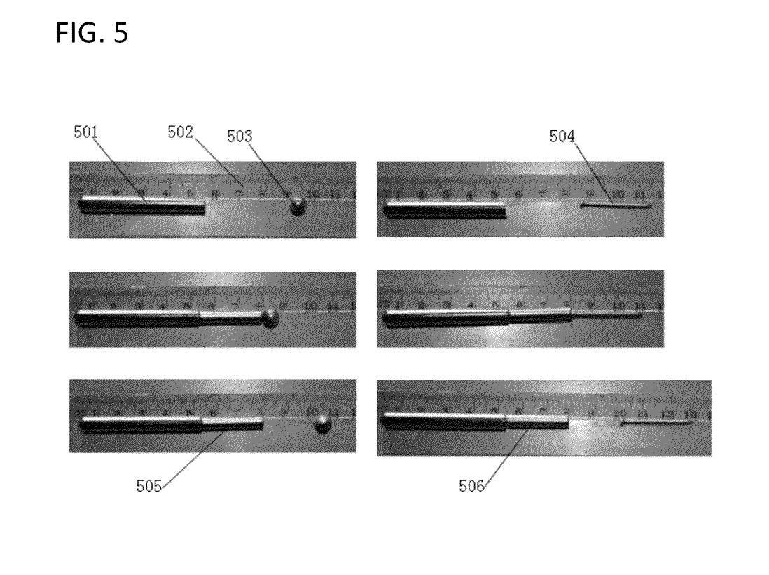

FIG. 5 shows an experiment that magnetic field can be transferred from magnet 501 to the soft magnetic collector to reach the target. The upper shows the iron ball 503 could not be captured in the distance between the permanent magnet and iron ball (indicated the distance by a ruler 502); The middle shows that by adding a soft magnetic collector 505 results the soft magnetic collector binds to magnet and the target iron ball binds to the soft magnetic collector; The lower shows that it has to increase the distance between the magnet and magnetic target to keep them separated (not bind together). Right side shows the magnet binds to the soft magnetic collector 506 and then the nail 504 binds to the soft magnetic collector.

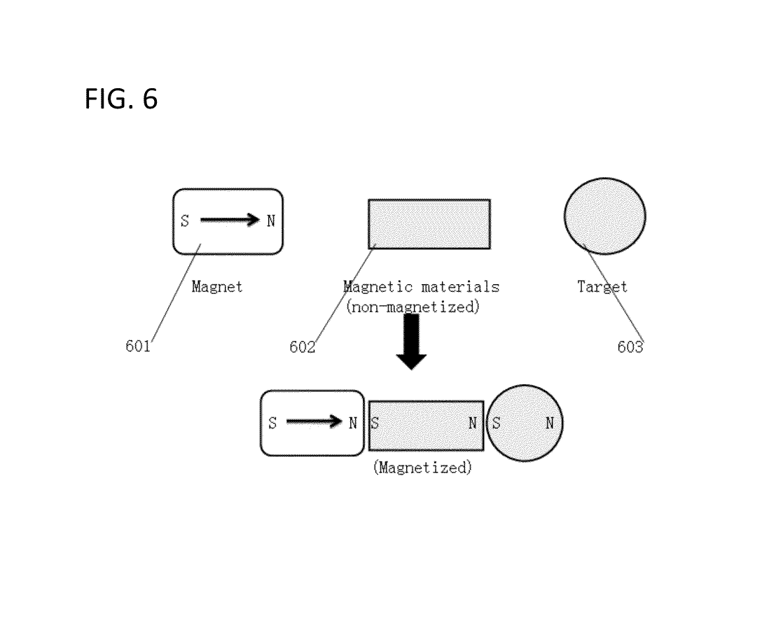

FIG. 6 describes the mechanism of magnetic field activation of soft magnetic collectors 602 that can let the magnetic target 603 bind to the soft magnetic collectors (magnetic insert) and the soft magnetic collector binds to the magnet 601.

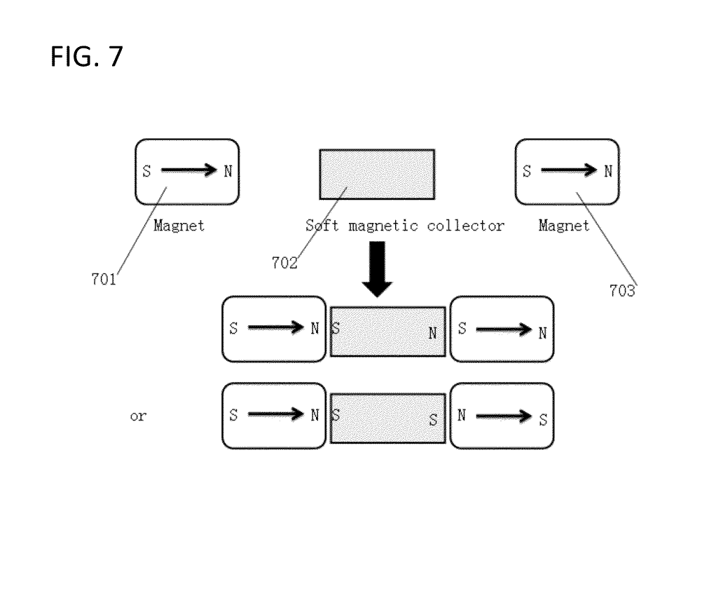

FIG. 7 describes the mechanism of a soft magnetic collector 702 can be bound between two magnets 701/703 with different magnetic field direction.

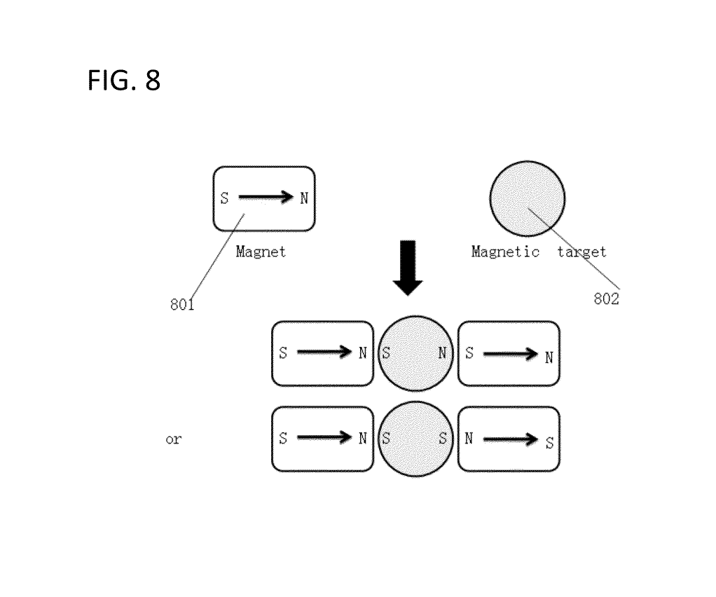

FIG. 8 describes the mechanism of the invention that the magnetic target 802 can bind by two magnets 801/802 in different magnetic field directions.

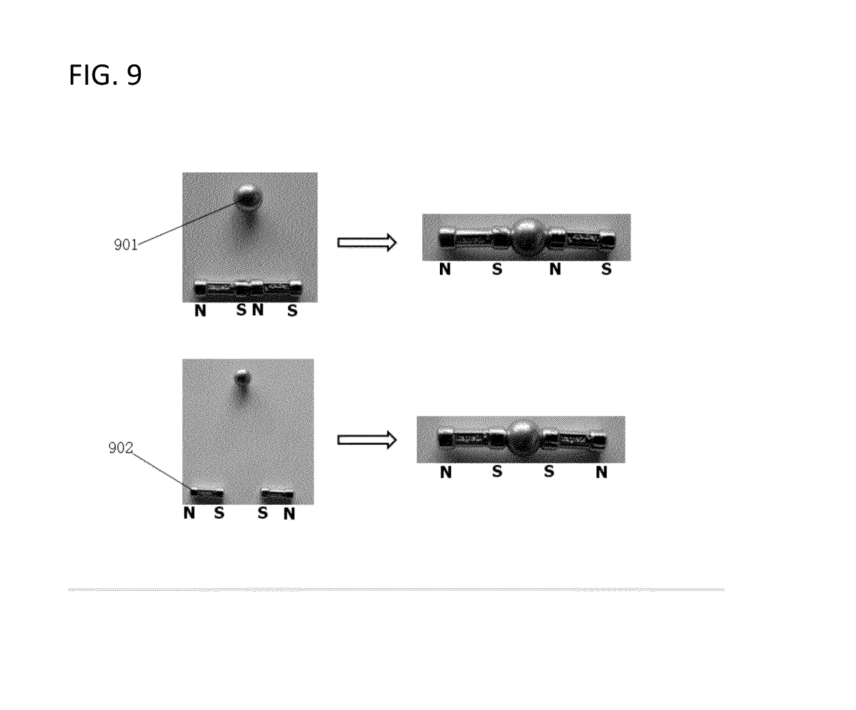

FIG. 9 shows an experiment that an iron ball 901 can be bound to two permanent magnets 902 in different magnetic field directions.

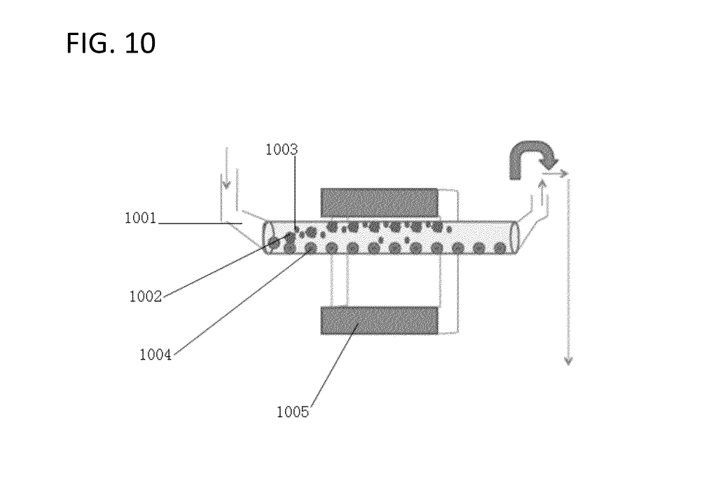

FIG. 10 illustrates a method to enrich and isolate target cells but remove the non target cells. The tube/channel 1001 at upper side, the target cells 1002 and some magnetic nano-particles 1003 will be captured, but the non target cells 1004 will be removed. The magnetic field can be controlled by electromagnet 1005 system.

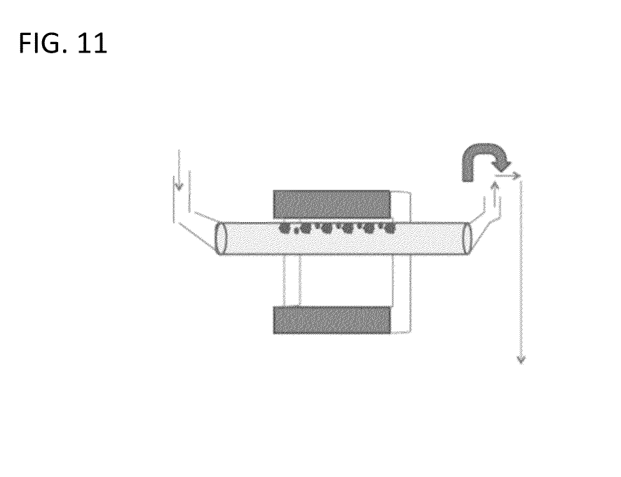

FIG. 11 illustrates the status of target cells and free magnetic nano-particles after first washing using a method to enrich and isolate target cells and remove the non target cells.

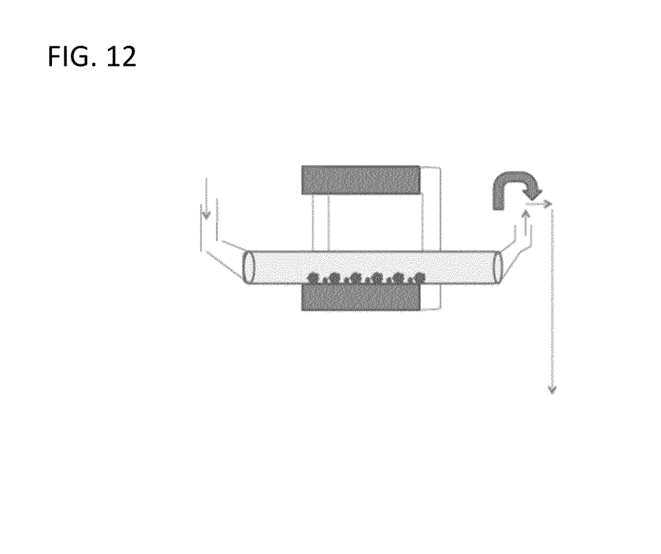

FIG. 12 illustrates a method to enrich and isolate target cells and remove the non target cells by placing the plastic tube near the lower magnet side.

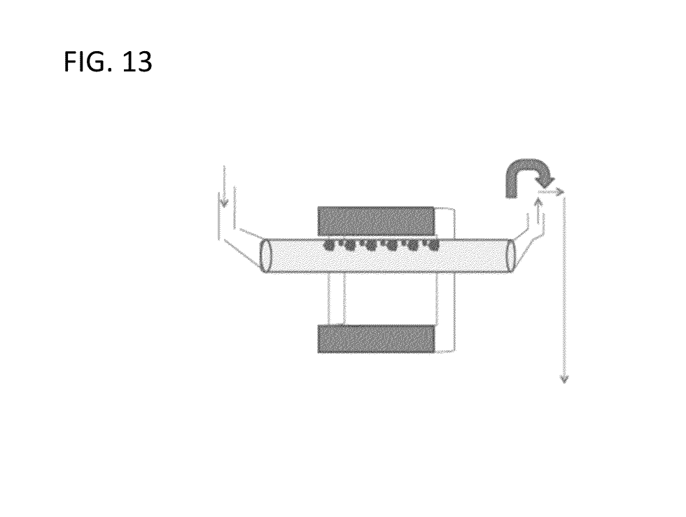

FIG. 13 illustrates a method to enrich and isolate target cells and remove the non target cells by placing the tube/channel to the upper magnetic pole to reduce the non target trapping.

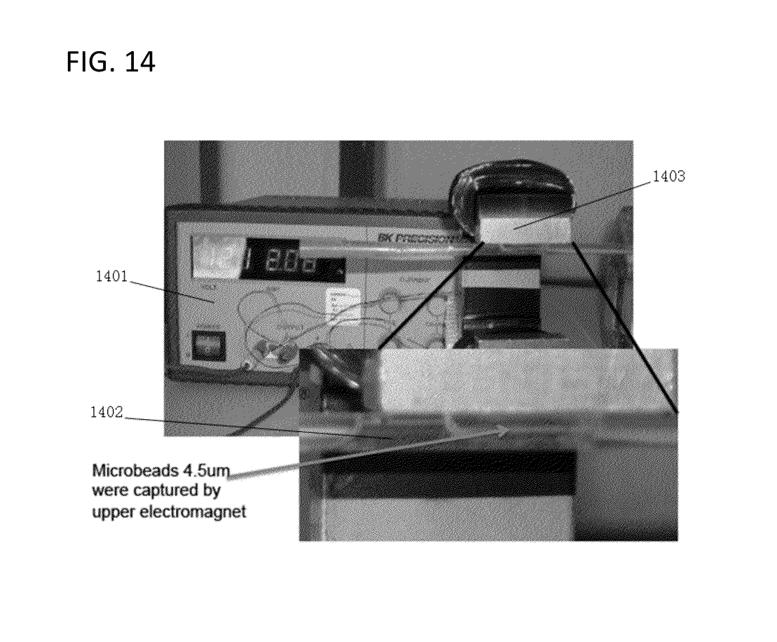

FIG. 14 shows an electromagnet device to isolate magnetic nano-particle labeled target cells. The target cells and free magnetic nano-particles will be captured, but non magnetic labeled biologicals will be removed. 1401 is the power supply for generating electromagnet 1403. 1402 is the tube/channel for target cells capturing.

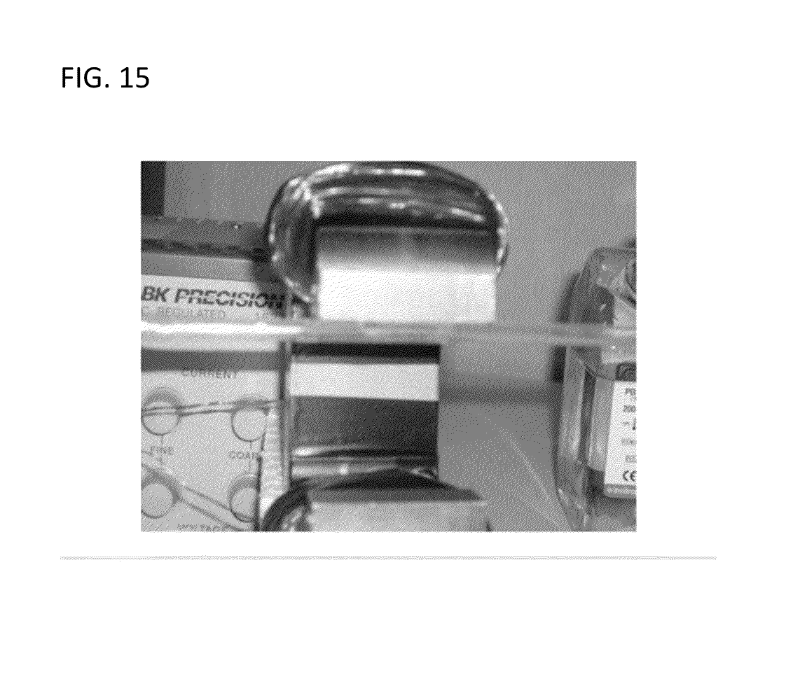

FIG. 15 shows the observation of the captured target cells using an electromagnet system.

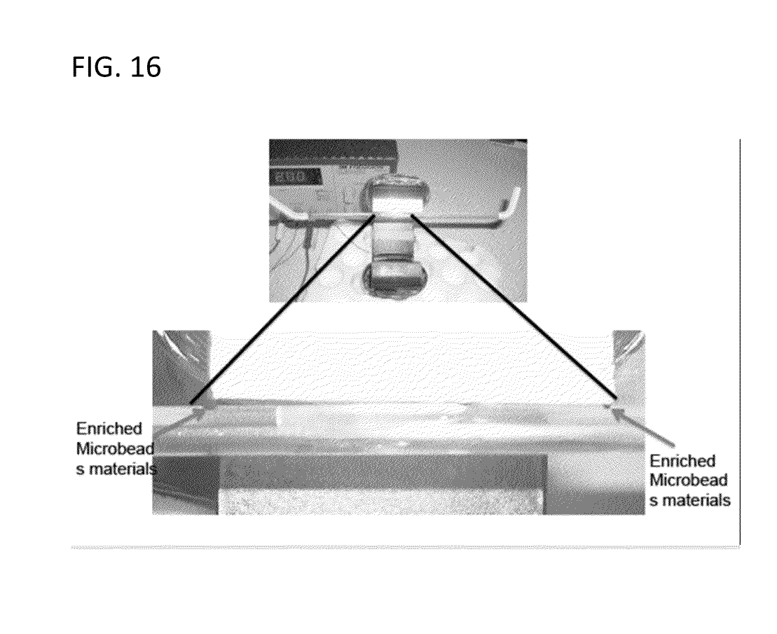

FIG. 16 shows the observation of an experiment that target cells were captured most in both end sides of the magnetic field in the plastic tube using a electromagnet device.

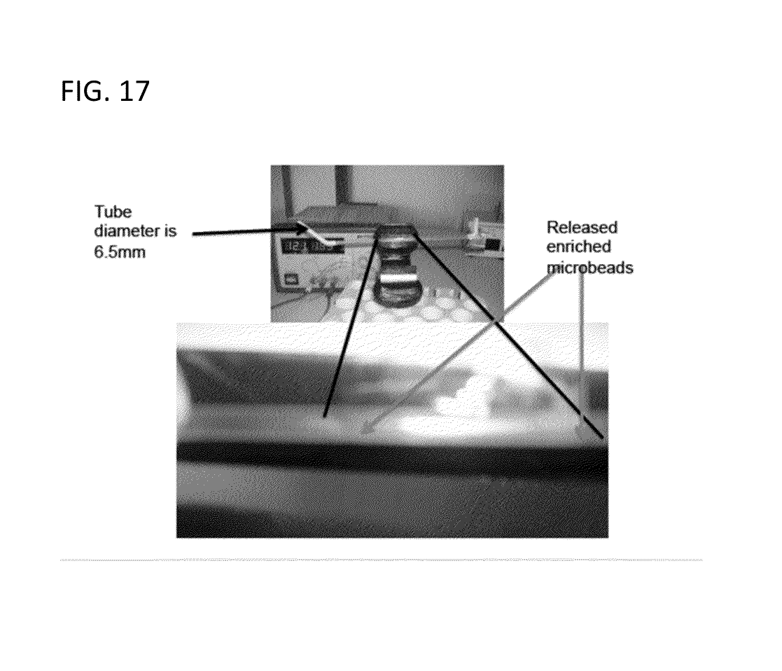

FIG. 17 shows the observation of captured targets using the electromagnet system.

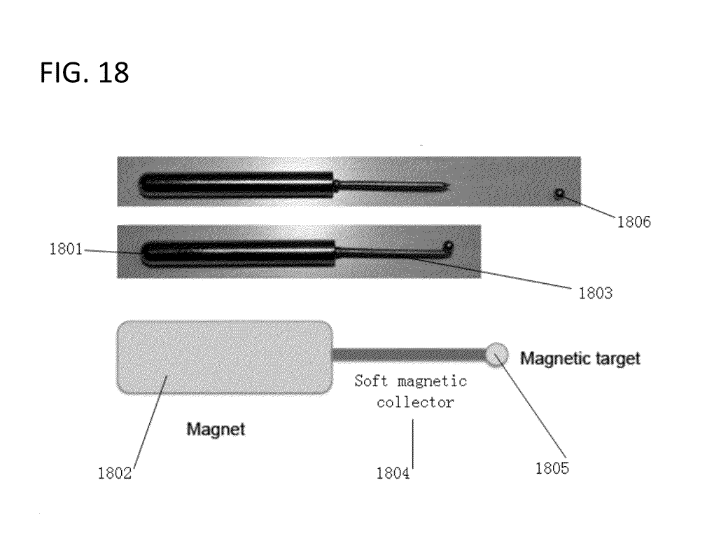

FIG. 18 shows an experiment using a schematic design to transfer the magnetic field from large magnet 1801/1802 to a small soft magnetic collector 1803/1804 to capture the magnetic targets 1805/1806. The magnet can reach the target iron ball by inserting a soft magnetic collector between.

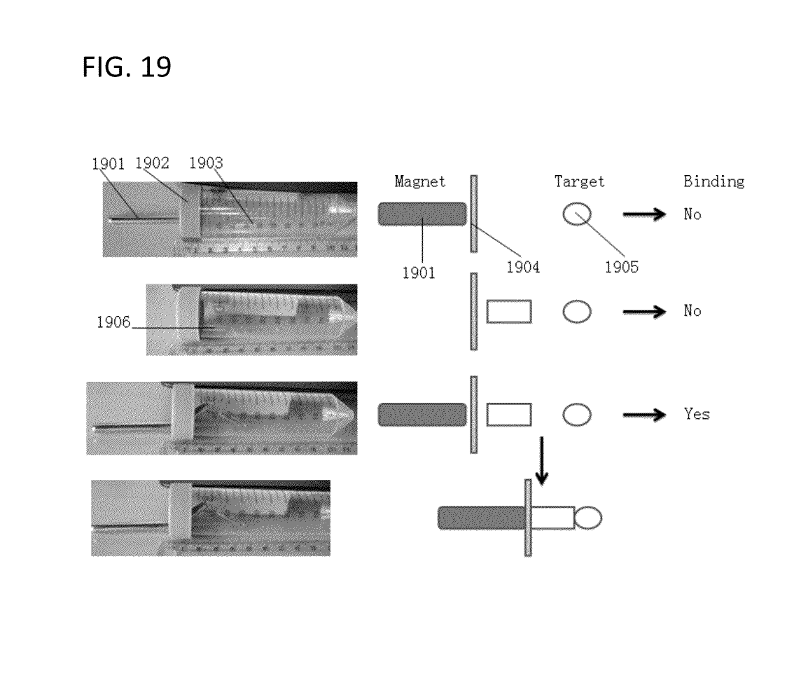

FIG. 19 shows an experiment that magnetic field of the magnet 1901 can go through the tube wall 1902/1904 and transfer to the soft magnetic collector 1906 to capture the target 1903/1905. Without the soft magnetic collector inside between the magnet and target, in a certain distance and with the tube wall, the target may not be captured, by adding an insert soft magnetic collector between the magnet and target, the target can be captured and increased the sensitivity than that without insert.

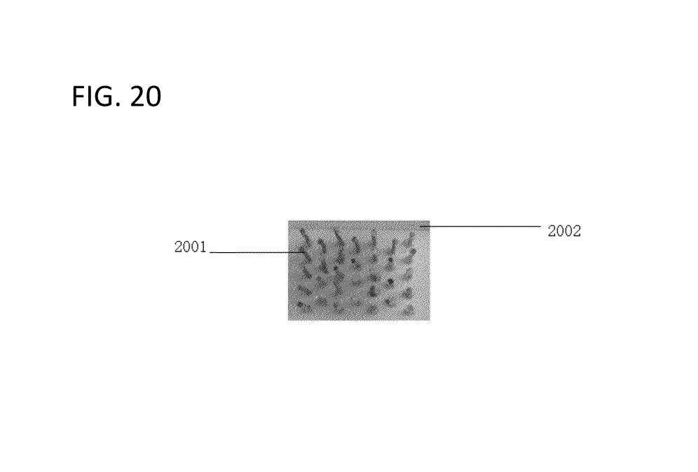

FIG. 20 illustrates a design of MagCell-CHIP that the magnetic posts as soft magnetic collectors 2001 inside of the container (box) 2002, the treated biological samples enter the CHIP, so the target cells will be bound to the magnetic posts under certain level of the magnetic field charge (magnetized), the non target cells will be removed.

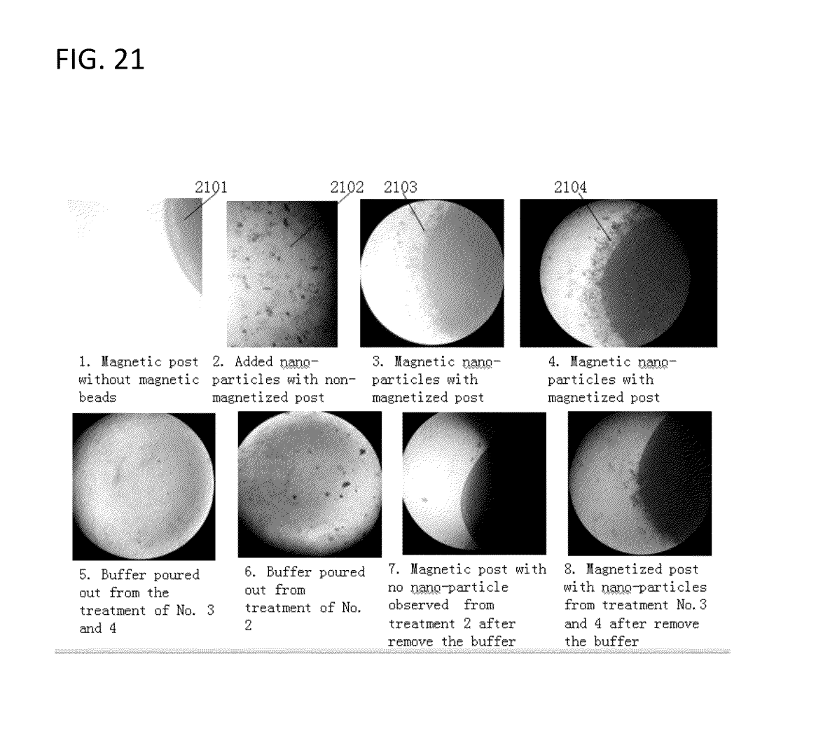

FIG. 21 shows the work results of the MagCell-CHIP for capturing or releasing the targets. The magnetic targets 2102 was added to the CHIP, after washing with the BPS buffer, the magnetic targets 2103/2104 bind to magnetic posts 2101 was observed. There are no magnetic targets from washing buffer, the magnetic targets can be released from the posts by turning off the magnetic field (FIG. 21-6).

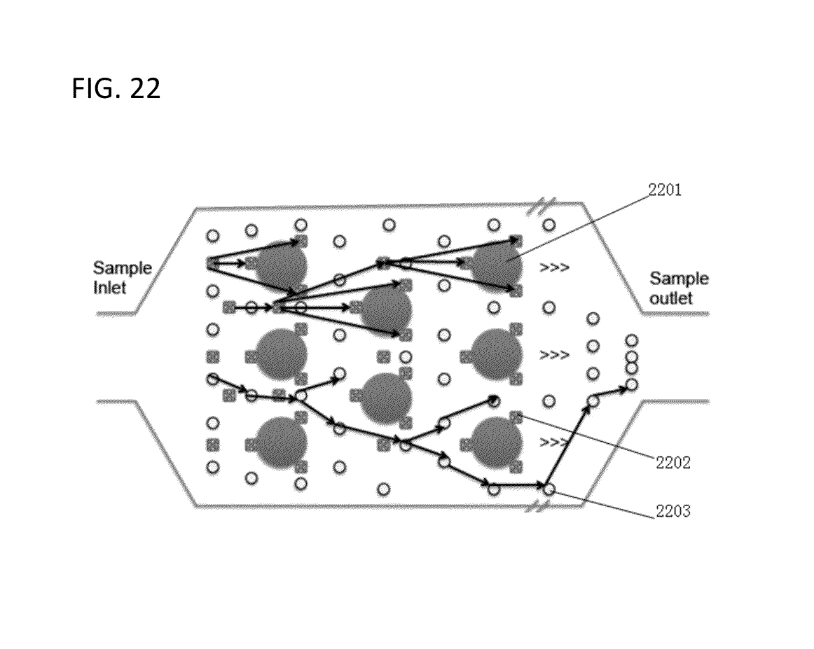

FIG. 22 illustrates the working schemes of the MagCell-CHIP. Under the magnetized posts, the magnetic targets 2202 will binds to the magnetic posts 2201, the non nano-particle labeled materials (non targets) 2203 will be removed from the CHIP.

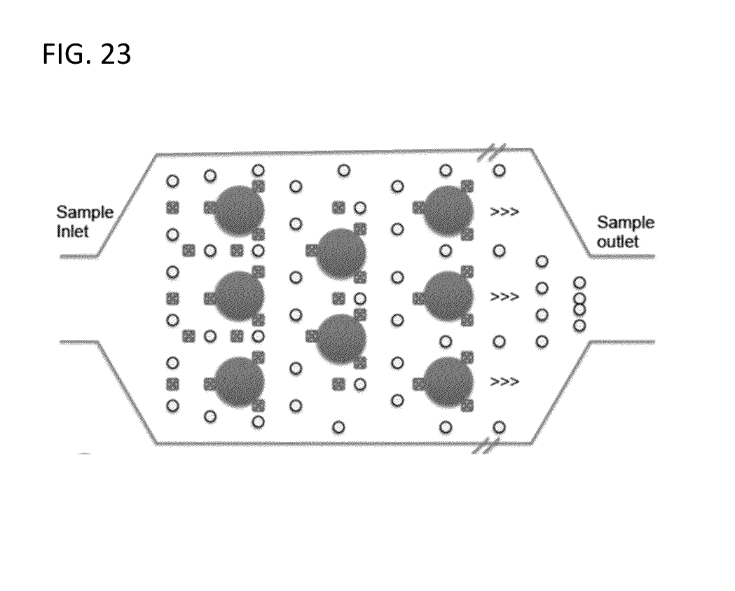

FIG. 23 illustrates the procedure that the target cells will be captured and the non target cells will be removed using the MagCell-CHIP.

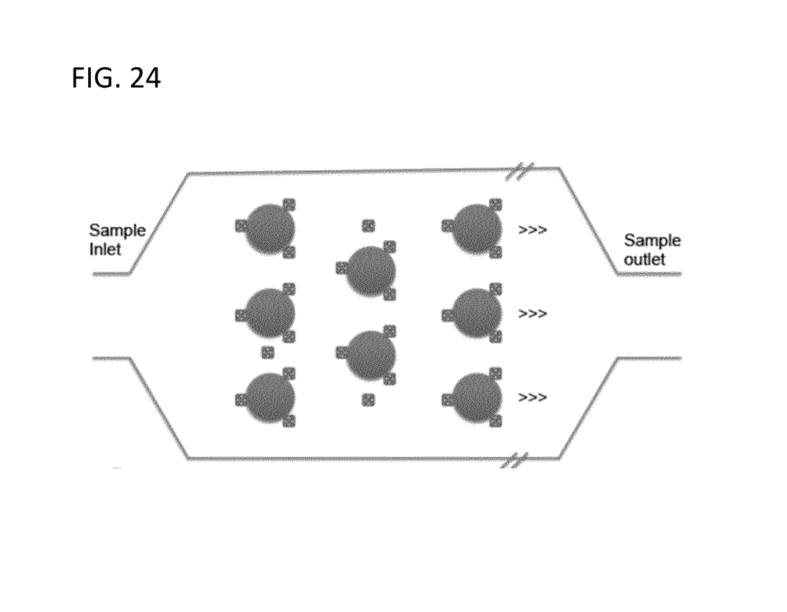

FIG. 24 demonstrates the pure target cells are captured by the soft magnetic posts after washing process using the MagCell-CHIP.

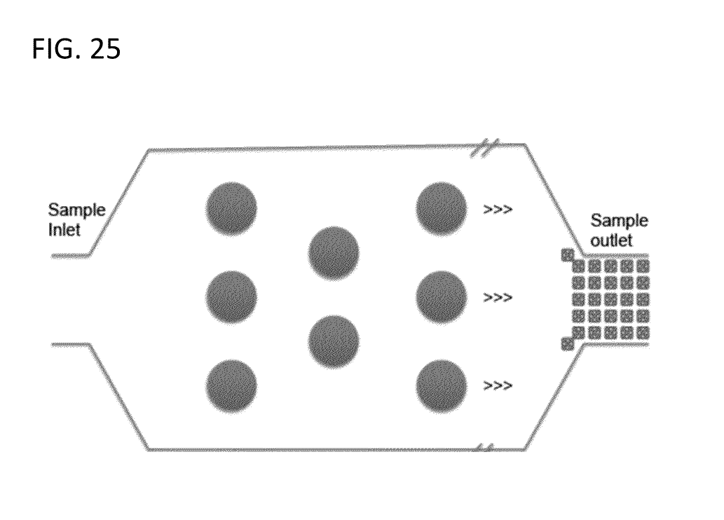

FIG. 25 shows the target cells can be removed from the soft magnetic posts after turning off the magnetic field using the MagCell-CHIP.

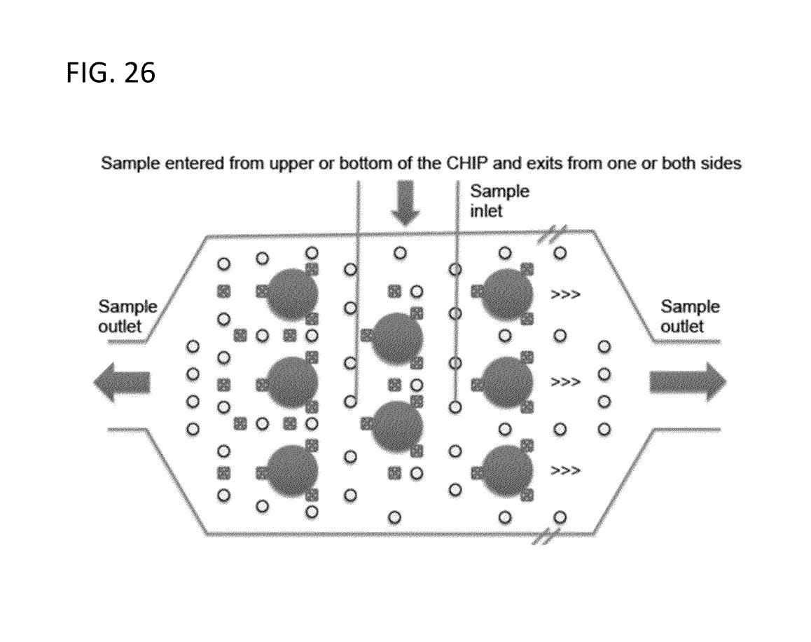

FIG. 26 illustrates a MagCell-CHIP that the sample can be added from upper side to the CHIP.

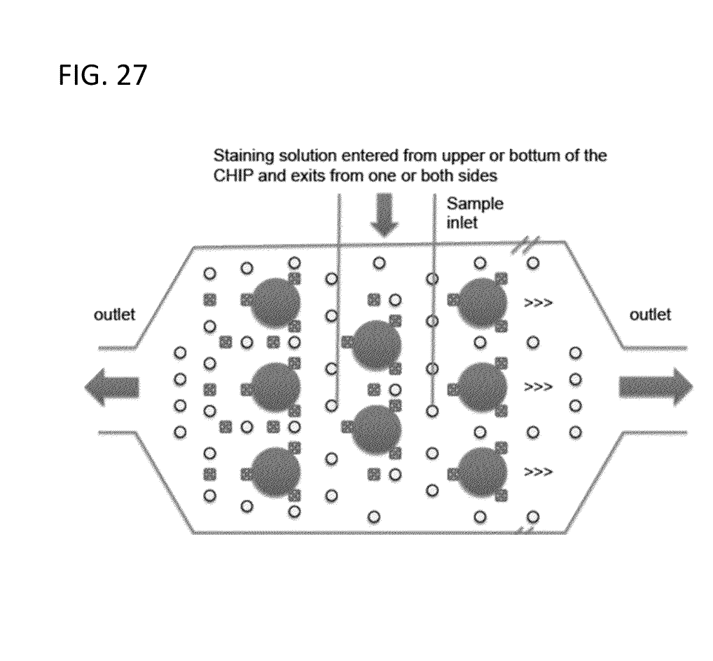

FIG. 27 illustrates the MagCell-CHIP can be used for the captured target cells staining or fixation within the MagCell-CHIP.

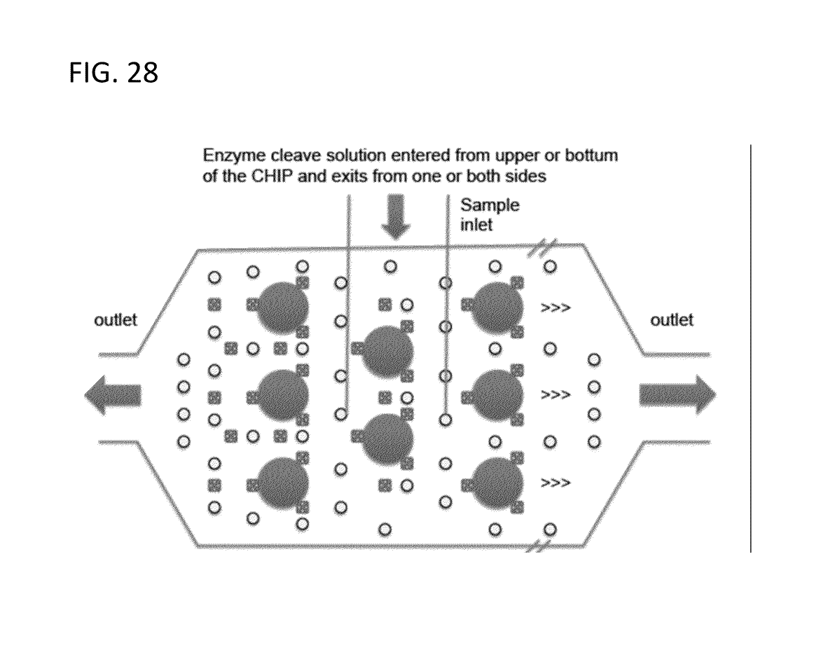

FIG. 28 illustrates the MagCell-CHIP can also be used for enzyme treatment of the captured target cells to remove any linkers between the magnetic beads and the target cells inside of the MagCell-CHIP.

### DETAILED DESCRIPTION OF THE INVENTION

The present invention is directed to the method and apparatus of isolating biological materials using a magnetic device that transfers the magnetic field from magnet to soft magnetic collector(s) and reaches to the magnetic targets. The device described here are for enrichment, isolation, and purification of targets such as magnetic labeled cells, DNA/RNA, protein and other biological materials. The device can be used for rare cell isolation and purification for various types of samples and complete the entire cell capturing, washing, and releasing cycle, or multiple rounds of the cycles through out the entire process, it is also can include the cell fixation, staining for cell identification, or cell sorting for downstream analyses. The advantage of such device in providing high sensitivity and isolate high purity targets, they can be used in other applications as well, such as pharmaceutical and food industry, or other industries where small quantity of material needs to be detected or analyzed.

### Embodiment #1

FIG. 1 illustrates the mechanism that by adding a soft magnetic collector between magnet and magnetic targets improved the magnet binding capability. Keeping the magnet and magnetic target a distance that magnet can not bind the target, but after adding the soft magnetic collector, the magnet can binds a soft magnetic collector and the magnetic target then binds to the soft magnetic collector. The magnet can be permanent magnet or electromagnet, the soft magnetic collector can be any magnet sensitive metal (iron, co, ni etc) and the target can be any magnetic labeled or magnetic sensitive material. FIG. 1 shows the magnetic field can be transferred from magnet to the inserted soft magnetic collector and reaches to target iron ball, the target capturing sensitivity is increased. This is improved by experiment in FIG. 5, FIG. 18 and be used in the cases of FIG. 2, FIG. 6, FIG. 7, FIG. 8 that the source magnet can transfers the magnetic field to a soft magnetic collector and increases the sensitivity to reach the magnetic targets.

### Embodiment #2

FIG. 19 shows an experiment and illustrates the magnetic field transferred from the source magnet through the tube well to a soft magnetic collector and reaches the magnetic target. The source magnet can not binds the pin inside of the tube because the distance (upper), also the pin does not move if there is no magnetic field charged (second). Keeping the pin away at the same distance (as in the upper case) from the source magnet and adding a soft magnetic collector, the binding has happened that the soft magnetic collector binds to the source magnet and the magnetic target binds to the soft magnetic collector (lower). After removing the source magnet, the soft magnetic collector and magnetic target will separate from each other. This describes that the source magnet can capture the magnetic target through the soft magnetic collectors. This can be used in the cases of FIG. 20 (a MagCell-CHIP), FIG. 21, FIG. 22, FIG. 23, FIG. 24, FIG. 25, FIG. 26, FIG. 27, FIG. 28 for capturing magnetic targets in a container.

### Embodiment #3

FIG. 2 illustrates an electromagnet captures magnetic targets inside of plastic tube that once the electromagnet is on, the magnetic labeled target cells will be captured, the non magnetic labeled cells will be removed. This can be used in the cases of FIG. 2-4, FIGS. 10-13 and it was improved by experiment in FIG. 14-17 that the magnetic labeled target cells are captured by the electromagnet efficiently.

### Embodiment #4

FIG. 22 illustrates the MagCell-CHIP functions that biological samples enter into the CHIP, the target cells will be captured by the magnetic posts which are activated by outside magnet, the non target cells and other materials will be removed. This is improved by the experiment in FIG. 20 and FIG. 21 that magnetic targets can be captured, released efficiently by the MagCell-CHIP device. This can be used in the cases in FIG. 22-28 for target cell enrichment, isolation, purification, fixation and staining.

### Other Embodiments

This invention using soft magnetic collectors for increasing targets capturing can be used in capturing cells by column and fluidics for targets isolation as well as for targets purification, transferring and biological analysis. The magnet, soft magnetic collector of the insert, the magnetic targets can be different size, direction and binding arrangements as shown in FIG. 6-8 and are improved in the experiments in FIG. 9.

General Procedures of MagCell-CHIP for Target Cell Enrichment, Isolation, Enzyme Treatment, Staining and Fixation

Target cells are labeled with magnetic nano-particles conjugated with specific antibody in separate plastic tube. The labeled sample contained the target cells is added into the MagCell-CHIP, pass the magnetic posts area with magnetic field charged, or incubate with rotating, the target cells will be bound to the magnetic posts. Adding the washing buffer (example: BPS buffer) to the MagCell-CHIP and wash it several times, the pure targets will be staying with the magnetic posts. To increase the purity, additional step of release the targets and re-capture it can be repeated till desired results obtained. From this step, the steps of the enzyme treatment, staining and fixation can be added here once or repeatedly to obtain desired results under magnetic field charged. The obtained targets can be collected by precipitation or centrifugation for downstream analyses.

If the targets were not labeled with magnetic nano-particles, then the magnetic nano-particles conjugated with specific antibodies can be added to the MagCell-CHIP, mixing well and turn on the magnetic field charges so the antibody conjugated nano-particles will be equally bound to the magnetic posts. Slowly let the fresh biological sample (liquid phase) passes the magnetic posts area, the target cells also can be captured for biological analyses.

### EXAMPLES

Examples below are for the purpose of providing references in demonstrating the principle of this invention only. The subject matter is not limited to these examples.

### Example 1

Target Iron Ball and Nail can be Captured by Magnet through Inserted a Soft Magnetic Collector.

FIG. 5 shows an iron ball in the distance (9 cm away from the end of magnet) to magnet that can not be captured by magnet, but by adding the soft magnetic collector, the same iron ball in the same distance was captured. To keep the iron ball separate from the complex of magnet and the soft magnetic collector, the distance has to increase to 10.5 cm away from the end of the source magnet (left). In the right side, this principle was demonstrated using nail instead of using iron ball. The example demonstrated the principle that source magnet can increase the sensitivity by adding a soft magnetic collector to reach magnetic target.

### Example 2

Electromagnet Captures Target Cells in the Plastic Tube in the Magnetic Field.

FIG. 14-17 shows that the electromagnet generates the magnetic field can efficiently capture the target cells in the plastic tube placed in the magnetic field area. The plastic tube with PBS buffer was placed in the magnetic field, the magnetic labeled target cells, once passing the plastic tube, it was captured in the plastic tube by the magnetic field force.

### Example 3

Magnet can through Plastic Tube Well, a Soft Magnetic Collector and Reaches the Magnetic Target

FIG. 19 shows the magnet can through plastic tube well, a soft magnetic collector and reaches the magnetic target. Placing a pin inside of the plastic tube with a distance and tight the cap, the outside magnet can not reach the pin (which means it could not bind the pin). Opening the cap, adding a piece of a soft magnetic collector (iron rod) behind the cap inside of the tube and tight the cap. Once place the magnet to the outside of the cap, the sot magnetic collector will binds to the magnet and the magnetic target also binds to the soft magnetic collector, the outside magnet reached the target through a soft magnetic collector passing the plastic cap well.

### Example 4

MagCell-CHIP Captures Magnetic Targets Efficiently.

FIG. 22 shows the MagCell-CHIP working process and FIG. 20 shows the MagCell-CHIP. Biological sample containing magnetic labeled cells added into the MagCell-CHIP with outside magnetic field charged, the sample inside of the MagCell-CHIP mixing well, so the target cells will binds to the magnetic posts (soft magnetic collectors). Adding washing buffer (example: BPS buffer) to wash off the non specific binding materials and the remains of the sample, so the target cells will be purified. Removing the outside magnet or turning off the magnetic field, the targets bound to the magnetic posts will be released and can be collected easily by simple precipitation of centrifugation for downstream analyses (observation, cell fixation, staining, cell collection for molecular analyses).

### REFERENCES

1. Nagrath S et al (2007): Isolation of rate circulating tumor cells in cancer patients by microchip technology. Nature 450: 1235-9.

2. Talasaz, A H et al (2009): Isolating highly enriched populations of circulating epithelial cells and other rare cells from blood using a magnetic sweeper device. PNAS USA 106: 3970-75.

3. Stott S L et al (2010): Isolation of circulating tumor cells use microvortex-generating herringbone-chip. PNAS USA 107: 18392-97.

1. A new soft magnetic collector isolation method using soft magnetic collector(s) to transfer magnetic field from magnet (permanent or electronic magnet source) directly (no insert or wall materials between the magnet and the soft magnetic collector) or indirectly (some insert or wall materials between the magnet and the soft magnetic collector) to the soft magnetic collector(s) and increase the interaction between targets and magnetic field (for example: bind the target for enrichment, isolation) comprising:
(a) Permanent or electronic magnet source materials to create magnetic field.
(b) Target collector for target materials collection.
(c) Soft magnetic collectors for transferring the magnetic field from magnet source to the soft magnetic collector(s) for magnetic targets directly bind to the soft magnetic collector(s) for target isolation or purification or enrichment.
(d) The targets including any magnetic particles or magnetic particle labeled biological materials.
(e) Capturing targets by the soft magnetic collector(s) or releasing the targets from the soft magnetic collector(s) controlled by magnetic field ON or OFF (moving away the magnet for permanent magnet, or turning the electromagnet OFF of moving away from magnet sensitive materials) system.
(f) Collect the targets from target collector by centrifugation.

2. The materials used to create magnetic field in claim 1, including permanent magnet or electromagnet which can be controlled to active or inactive the magnetic field as needed.

3. The target material collector in claim 1, including tubes, plates or fluidics and chips or other container for target material incubation, mixing, fixation, staining and other collection use.

4. The soft magnetic collector(s) in claim 1, including any kind of soft magnetic materials (iron, co, ni) or metal coated with desired shape and strength, position fixed or floating in the target collector.

5. The target isolation method as in claim 1, the target capturing including mixing, shaking, electricity charging or passing the soft magnetic material's magnetic field to make the efforts for binding the targets to the soft magnetic collector(s) when it activated.

6. The target isolation method as in claim 1, including a target purification step which is to add a washing step during the target isolation procedures. This washing step can be done once or multiple time, or bind and release the target for multiple washing with wash buffers.

7. The target isolation method as in claim 1, including option step for target labeling with different reagents or fixation. This step can be done when the targets bind to the magnet sensitive materials or released from the magnet sensitive materials and re-capture it later.

8. The target isolation method as in claim 1, including a step for target collection by centrifugation so the target can be collected together or individually for further analysis.

9. The method includes the soft magnetic collector(s) or magnetic labeled biological materials pass the magnetic field once or multiple times for capturing and purification purposes.

10. The source magnet in claim 1 including permanent magnet and electromagnet. The container of biological samples can placed in the magnetic field for target enrichment, isolation and collection.

11. One use of the invention in claim 10 is making MagCell-CHIP or MagCell-Fluidics. This device is using magnetic field ON/OFF to operate the targets capturing, washing, releasing and collection, also for target fixation, staining and observation purposes.

12. The MagCell-CHIP in claim 11 is using magnetic structure inside of a container for magnetic targets directly bind to the soft magnetic collector structure(s) to increase the capture sensitivity, equally capture the targets in the structure.

13. The MagCell-CHIP in claim 11 has the advantage to observe the cells under microscope or other equipment. Once the outside magnetic field is turned OFF, the captured target cells will be released on the surface of the bottom wall of the container, it is easy for morphological observation and microscopic scanning.

14. The MagCell-CHIP in claim 11 has the advantage to stain the targets inside of the container. Once the targets captured by the MagCell-CHIP, the target cells should be pure after washing step. These targets can be stained in the container by adding staining solution, release the cells from the posts, mixing well and finishing the staining and re-capture the targets and switch for other staining if needed. After releasing the stained, captured targets, it will be easier for morphological observation.

15. The MagCell-CHIP in claim 11 has the advantage to fix the targets as needed. The staining process can be done when the targets were bound to the magnetic posts, or during the targets released or the combination of captured and released.

16. The MagCell-CHIP in claim 11 can be various in sizes, shapes, colors for different sample volume processing. It can be one in a magnetic field or many in a magnetic field to increase the assay capability.

17. The MagCell-CHIP in claim 11, the soft magnetic collector structure inside of the container can be different structure. The posts can be different shape, the distance between each posts can be different and be arranged, the structure can be with posts, or pieces of magnetic metal attached one side or multiple sides of the container.

18. The MagCell-CHIP in claim 11 can be rotated once operating with the samples in the magnetic field. The operation process can be automated by a device such as robotic arms.

19. The unbound free magnetic particles inside of the container can be filtered with additional device such as filter.

20. Measure the bound targets inside of the MagCell-CHIP in claim 11 by magnetic sensor device with the MagCell-CHIP or collected and then be measured for enumeration purposes.
